(19) United States
(12) Patent Application Publication
(10) Pub. No.: US 2008/0058299 A1
(43) Pub. Date: Mar. 6, 2008

(54) Method of treating or preventing bone deterioration or osteoporosis

(76) Inventor: Robert E. Dudley, Rosemary Beach, FL (US)

Correspondence Address: MAYER BROWN LLP, P.O. BOX 2828, CHICAGO, IL, 60690, US

(21) Appl. No.: 11/402,682

(22) Filed: Apr. 11, 2006

Related U.S. Application Data
(60) Provisional application No. 60/670,580, filed on Apr. 12, 2005.

(51) Int. Cl. A61K 31/568; A61P 19/10

(52) U.S. Cl. 514/178

(57) ABSTRACT

The present invention relates to a transdermal hydroalcoholic testosterone gel formulation and a method for treating, preventing, or reducing the risk of developing deterioration of bone. The present invention also relates to a method for treating, preventing, or reducing the risk of developing osteoporosis.

This application claims priority to U.S. provisional Application Ser. No. 60/670,580 filed Apr. 12, 2005, the entire contents of which is hereby incorporated by reference herein.

### FIELD OF THE INVENTION

The present invention is generally related to a method of treating, preventing, or reducing the risk of developing bone deterioration or osteoporosis, and, more particularly, is related to a method of administering a transdermal hydroalcoholic gel composition to treat or prevent bone deterioration or osteoporosis.

### BACKGROUND OF THE INVENTION

Osteoporosis is a significant public health problem in men as well as in women. Vertebral fracture rates over age 50 are as high in men as in women. Jackson S A, et al., Osteoporosis Int., 11:680-7 (2000); O'Neill T W, et al., J. Bone Miner. Res., 11:1010-8 (1996). Although the hip fracture rate in men is only half that in women, Mussoline M E, et al., J. Bone Miner. Res. 13: 918-24 (1998); Kellie S E, et al., Am J. Public Health, 80:326-8 (1990), the mortality after hip fracture in men is double that in women. Kanis J A, et al., Bone, 32:468-73 (2003). Osteoporosis is characterized by the loss of bone and deterioration of the trabecular architecture. The normally interdigitating plates become fenestrated and eventually become rods, and then the rods become disconnected, leading to decreased strength and increased tendency to fracture. Parfitt A M, Bone, 13:S41-7 (1992); Parfitt A M, et al., J. Clin. Invest., 72:1396-409 (1983).

Severe hypogonadism is a well-documented cause of osteoporosis in men, as in women. Men who are severely hypogonadal due to pituitary or testicular disease have lower bone mineral density (BMD) than eugonadal men, as well as deteriorated trabecular architecture. Katznelson L, et al., J. Clin. Endocrinol. Metab., 4358-4365 (1996); Devogelaer J P, et al., Maturitas:17-23; Benito M; et al., J. Clin. Endocrinol. Metab., 85:2670-7 (2000).

Testosterone, the major circulating androgen in men, is synthesized from cholesterol. The approximately 500 million Leydig cells in the testes secrete more than 95% of the 6-7 mg of testosterone produced per day. Two hormones produced by the pituitary gland, luteinizing hormone (“LH”) and follicle stimulating hormone (“FSH”), are required for the development and maintenance of testicular function and negatively regulate testosterone production. Circulating testosterone is metabolized to various 17-keto steroids through two different pathways. Testosterone can be metabolized to dihydrotestosterone (“DHT”) by the enzyme 5α-reductase or to estradiol (“E2”) by an aromatase enzyme complex.

Testosterone circulates in the blood 98% bound to protein. In men, approximately 40% of the binding is to the high-affinity sex hormone binding globulin (“SHBG”). The remaining 60% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-SHBG bound testosterone and includes testosterone weakly bound to albumin.

The following table from the UCLA-Harbor Medical Center summarizes the hormone concentrations in normal adult men range:

TABLE 1
Hormone Levels in Normal Men

| Hormone | Normal Range |
|---|---|
| Testosterone | 298 to 1043 ng/dL |
| Free testosterone | 3.5 to 17.9 ng/dL |
| DHT | 31 to 193 ng/dL |
| DHT/T Ratio | 0.052 to 0.33 |
| DHT + T | 372 to 1349 ng/dL |
| SHBG | 10.8 to 46.6 nmol/L |
| FSH | 1.0 to 6.9 mlU/mL |
| LH | 1.0 to 8.1 mlU/mL |
| E₂ | 17.1 to 46.1 pg/mL |

There is considerable variation in the half-life of testosterone reported in the literature, ranging from 10 to 100 minutes. Researchers do agree, however, that circulating testosterone has a diurnal variation in normal young men. Maximum levels occur at approximately 6:00 to 8:00 a.m. with levels declining throughout the day. Characteristic profiles have a maximum testosterone level of 720 ng/dL and a minimum level of 430 ng/dL. The physiological significance of this diurnal cycle, if any, however, is not clear.

Male hypogonadism results from a variety of patho-physiological conditions in which testosterone concentration is diminished below the normal range. The hypogonadic condition is sometimes linked with a number of physiological changes, such as diminished interest in sex, impotence, reduced lean body mass, decreased bone density, lowered mood, and decreased energy levels.

Researchers generally classify hypogonadism into one of three types. Primary hypogonadism includes the testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high FSH and LH concentrations. However, because of testicular or other failures, the high LH concentrations are not effective at stimulating testosterone production.

Secondary hypogonadism involves an idiopathic gonadotropin or LH-releasing hormone deficiency. This type of hypogonadism includes Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Third, hypogonadism may be age-related. Men experience a slow but continuous decline in average serum testosterone after approximately age 20 to 30 years. Researchers estimate that the decline is about 1-2% per year. Cross-sectional studies in men have found that the mean testosterone value at age 80 years is approximately 75% of that at age 30 years. Because the serum concentration of SHBG increases as men age, the fall in bioavailable and free testosterone is even greater than the fall in total testosterone. Researchers have estimated that approximately 50% of healthy men between the ages of 50 and 70 have levels of bioavailable testosterone that are below the lower normal limit. Moreover, as men age, the circadian rhythm of testosterone concentration is often muted, dampened, or completely lost. The major problem with aging appears to be within the hypothalamic-pituitary unit. For example, researchers have found that with aging, LH levels do not increase despite the low testosterone levels. Regardless of the cause, these untreated testosterone deficiencies in older men may lead to a variety of physiological changes, including sexual dysfunction, decreased libido, loss of muscle mass, decreased bone density, depressed mood, and decreased cognitive function. The net result is geriatric hypogonadism, or what is commonly referred to as “male menopause.” Today, hypogonadism is the most common hormone deficiency in men, affecting 5 in every 1,000 men. At present, it is estimated that only five percent of the estimated four to five million American men of all ages with hypogonadism currently receive testosterone replacement therapy.

In addition, most agents used to treat osteoporosis, such as estrogens and bisphosphonates, are not very effective. These agents retard bone resorption but do not improve connectivity, with the possible exception of recombinant human parathyroid hormone 1-34 (rhPTH, teriparatide). Vedi S, et al., Bone, 19:69-72 (1996); Borah B, et al., Bone, 34:736-46 (2004). Treatment of 32 postmenopausal women with rhPTH for an average of 19 months significantly increased their trabecular connectivity compared to that of 19 placebo-treated women. Jiang Y, et al., J. Bone Miner. Res., 18:1932-41 (2003). Unlike physiologic replacement of testosterone in hypogonadal men or women, the rhPTH treatment was pharmacologic, and involved administration of a hormone to women not deficient in that hormone.

Thus, there is a need in the art for a safe and effective treatment for treating, preventing, or reducing the risk of developing bone disorders, conditions, or diseases, such as, e.g., bone deterioration or osteoporosis.

### SUMMARY OF THE INVENTION

The present invention relates to a transdermal hydroalcoholic testosterone gel formulation and a method for treating, preventing, or reducing the risk of developing deterioration of bone. The present invention also relates to a method for treating, preventing, or reducing the risk of developing osteoporosis.

### BRIEF DESCRIPTION OF THE DRAWINGS

FIG. 1 is a graft showing serum testosterone and estradiol concentrations when ten hypogonadal men were treated with testosterone transdermally for 24 months.

FIG. 2a is a graph showing changes in one of the principal composite magnetic resonance microimaging parameters, the surface-to-curve ratio of the distal tibia, when ten hypogonadal men were treated with testosterone transdermally for 24 months.

FIG. 2b is a graph showing changes in one of the principal composite magnetic resonance microimaging parameters, the topical erosion index of the distal tibia, when ten hypogonadal men were treated with testosterone transdermally for 24 months.

FIG. 3a is a cross-sectional image of the tibia of a hypogonadal subject before testosterone treatment.

FIG. 3b is a cross-sectional image of the tibia of a hypogonadal subject after 24 months of testosterone treatment.

FIG. 3c is a high resolution surface projection image of the tibia of a hypogonadal subject, before testosterone treatment. The surface projection image was taken from the circle area in FIG. 3a.

FIG. 3d is a high resolution surface projection image of the tibia of a hypogonadal subject after 24 months of testosterone treatment. The surface projection image was taken from the circle area in FIG. 3b.

FIG. 4 is a graph demonstrating the prevalence of hypogonadism in patients grouped by age

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated. Where the invention is illustrated herein with particular reference to testosterone, it will be understood that any other steroid in the testosterone synthetic pathway can, if desired, be substituted in whole or in part for testosterone in the methods, kits, combinations, and compositions herein described.

The present invention relates to a method for preventing, improving or treating bone disorders, conditions or diseases, such as, e.g., bone deterioration, including, for example, deterioration of the trabecular architecture, or osteoporosis. In one embodiment, the present invention is directed to a method for percutaneous administration of testosterone in a hydroalcoholic gel. The gel comprises one or more lower alcohols, such as ethanol or isopropanol; a penetration enhancing agent; a thickener (aka a gelling agent); and water. Additionally, the present invention may optionally include salts, emollients, stabilizers, antimicrobials, fragrances, and propellants.

The present invention also includes kits, methods, combinations, and pharmaceutical compositions for treating, preventing, reversing, halting or slowing the progression of bone disorders, conditions, or diseases, such as bone deterioration or osteoporosis in a subject once it becomes clinically evident, or treating the symptoms associated with, or related to the bone deterioration, such as, e.g., deterioration of the trabecular architecture, or osteoporosis. The subject may already have a diagnosis of a bone disorder, condition or disease, such as, e.g., bone deterioration or osteoporosis, at the time of administration, or be at risk of developing bone deterioration or osteoporosis.

The term “derivative” refers to a compound that is produced from another compound of similar structure by the replacement of substitution of one atom, molecule or group by another. For example, a hydrogen atom of a compound may be substituted by alkyl, acyl, amino, etc., to produce a derivative of that compound.

As used herein, the term “lower alcohol,” alone or in combination, means a straight-chain or branched-chain alcohol moiety containing one to about six carbon atoms. In one embodiment, the lower alcohol contains one to about 4 carbon atoms, and in another embodiment the lower alcohol contains two to about 3 carbon atoms. Examples of such alcohol moieties include methanol, ethanol, n-propanol, isopropanol, n-butanol, isobutanol, sec-butanol, and tert-butanol.

As used herein, the term “lower alkyl”, alone or in combination, means a straight-chain or branched-chain alkyl radical containing one to about six carbon atoms. In one embodiment, the lower alkyl contains one to about four carbon atoms. Examples of such radicals include methyl, ethyl, n-propyl, isopropyl, n-butyl, isobutyl, sec-butyl, and tert-butyl.

The phrase “penetration enhancing agent” refers to an agent that accelerates the delivery of the drug through the skin. These agents also are referred to as accelerants, adjuvants, and absorption promoters, and are collectively referred to herein as “enhancers.” This class of agents includes those with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer. The penetration enhancing agent of the present invention is a functional derivative of a fatty acid, which includes isosteric modifications of fatty acids or non-acidic derivatives of the carboxylic functional group of a fatty acid or isosteric modifications thereof. In one embodiment, the functional derivative of a fatty acid is an unsaturated alkanoic acid in which the —COOH group is substituted with a functional derivative thereof, such as alcohols, polyols, amides and substituted derivatives thereof. The term “fatty acid” means a fatty acid that has four (4) to twenty-four (24) carbon atoms.

The composition is used in a “pharmacologically effective amount.” This means that the concentration of the drug administered is such that in the composition it results in a therapeutic level of drug delivered over the term that the drug is to be used. Such delivery is dependent on a number of variables including the time period for which the individual dosage unit is to be used, the flux rate of the drug from the composition, for example, testosterone, from the gel, surface area of application site, etc. For testosterone, for example, the amount of testosterone necessary can be experimentally determined based on the flux rate of testosterone through the gel, and through the skin when used with and without enhancers.

The term “prodrug” refers to a drug or compound in which the pharmacological action (active curative agent) results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard [Int. J. Pharmaceutics, 37, 87 (1987)] describe the evaluation of N-acylsulfonamides as potential prodrug derivatives. J. Larsen et al., [Int. J. Pharmaceutics, 47, 103 (1988)] describe the evaluation of N-methylsulfonamides as potential prodrug derivatives. Prodrugs are also described in, for example, Sinkula et al., J. Pharm. Sci., 64:181-210 (1975). Other nonlimiting examples of “prodrugs” that can be used in the combinations and methods of the present invention include parecoxib (propanamide, N-[[4-(5-methyl-3-phenyl-4-isoxazolyl)phenyl]sulfonyl]-), and MAG-camptothecin.

In one embodiment, the present invention is directed to a method for percutaneous administration of testosterone in a hydroalcoholic gel. The gel comprises one or more lower alcohols, such as ethanol or isopropanol; a penetration enhancing agent; a thickener (aka a gelling agent); and water. In one embodiment, the gel further comprises a hydroxide releasing agent, such as, e.g, sodium hydroxide. Additionally, the present invention may optionally include salts, emollients, stabilizers, antimicrobials, fragrances, and propellants.

A class of steroids in the testosterone synthetic pathway useful in the methods and compositions of the present invention include steroids in the testosterone anabolic or catabolic pathway. In a broad aspect of the invention, the active ingredients employed in the present invention may include anabolic steroids such as androisoxazole, androstenedione, bolasterone, clostebol, ethylestrenol, formyldienolone, 4-hydroxy-19-nortestosterone, methenolone, methyltrienolone, nandrolone, oxymesterone, quinbolone, stenbolone, trenbolone; androgenic steroids such as boldenone, dehydroepiandrosterone, fluoxymesterone, mestanolone, mesterolone, methandrostenolone, 17 alpha-methyltestosterone, 17 alpha-methyl-testosterone 3-cyclopentyl enol ether, norethandrolone, normethandrone, oxandrolone, oxymetholone, prasterone, stanlolone, stanozolol, dihydrotestosterone, testosterone; and progestogens such as anagestone, chlormadinone acetate, delmadinone acetate, demegestone, dimethisterone, dihydrogesterone, ethinylestrenol, ethisterone, ethynodiol, ethynodiol diacetate, flurogestone acetate, gestodene, gestonorone caproate, haloprogesterone, 17-hydroxy-16-methylene-progesterone, 17 alpha-hydroxyprogesterone, 17 alpha-hydroxyprogesterone caproate, medrogestone, medroxyprogesterone, megestrol acetate, melengestrol, norethindrone, norethindrone acetate, norethynodrel, norgesterone, norgestimate, norgestrel, norgestrienone, 19-norprogesterone, norvinisterone, pentagestrone, prenenolone, progesterone, promegestone, quingestrone, and trengestone; and all salts, esters, amides, enantiomers, isomers, tautomers, prodrugs and derivatives of these compounds. (Based in part upon the list provided in The Merck Index, Merck & Co. Rahway, N.J. (1998)). Combinations of the above mentioned steroids can be used in the methods, kits, combinations, and compositions herein described.

Included in the methods and pharmaceutical compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diusopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickening agents, or gelling agents, used herein may include anionic polymers such as polyacrylic acid (CARBOPOL® by B.F. Goodrich. Specialty Polymers and Chemicals Division of Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol(® 954, Carbopol® 980, Carbopol(® 981, Carbopol® ETD 2001, Carbopol® EZ-2 and Carbopol® EZ-3, and other polymers such as Pemulen® polymeric emulsifiers, and Noveon® polycarbophils. Additional thickening agents, enhancers and adjuvants may generally be found in Remington's The Science and Practice of Pharmacy, Meade Publishing Co., United States Pharmacopeia/National Formulary.

In one embodiment, the formulation of the present invention delivers about 0.5 mg to about 250 mg testosterone, or the equivalent thereof, to a subject per dosage unit. In another embodiment of the present invention, the formulation delivers from about 5 mg to about 150 mg testosterone, or the equivalent thereof, to a subject per dosage unit. In yet another embodiment of the present invention, the formulations of the present invention deliver from about 25 mg to about 100 mg testosterone, or the equivalent thereof, to a subject per dosage unit. In another embodiment of the present invention, the formulations of the present invention deliver about 50 mg to about 100 mg testosterone, or the equivalent thereof, to a subject per dosage unit. In still another embodiment of the present invention, the formulations of the present invention deliver about 100 mg testosterone, or the equivalent thereof, to a subject per dosage unit. Thus, for example, a testosterone gel, ointment, cream or patch formulated for once a day administration can contain about 25 mg, or about 50 mg, or about 75 mg, or about 100 mg testosterone.

In one embodiment, the formulation is a gel, an ointment, a cream or a patch and is comprised of testosterone; a penetration enhancing agent, such as isopropyl myristate; a thickening agent, such as Carbopol; a lower alcohol, such as ethanol or isopropanol; and water. In another embodiment the formulation is a gel, an ointment, a cream or a patch and is comprised of the following substances in approximate percentages:

TABLE 2
Composition of Testosterone Formulation

| SUBSTANCE | AMOUNT (w/w) |
|---|---|
| Testosterone | 0.01-15% |
| Penetration | 0.01-50% |
| enhancing agent | |
| Gelling agent | 0.01-50% |
| Lower alcohol | 30-98% |
| Purified water (qs) | to 100% |

In one embodiment, in a 100 g composition, the gel, ointment, cream, or patch may contain about 0.01 g to about 15 g of testosterone, about 0.01 g to about 50 g penetration enhancing agent, about 0.1 g to about 50 g gelling agent, and about 30 g to about 98 g lower alcohol. In another embodiment, in a 100 g composition, the gel, ointment, cream, or patch may contain about 0.1 g to 10 g of testosterone, about 0.1 g to about 5 g of penetration enhancing agent, about 0.1 g to about 5 g of gelling agent, about 45 g to about 90 g lower alcohol, and the balance water.

In one embodiment, the composition is a gel, ointment, cream, or patch that further comprises sodium hydroxide or triethanolamine or potassium hydroxide, or a combination thereof, in an amount sufficient, as is known in the art, to assist the gelling agent in forming a gel. In one embodiment, a solution of sodium hydroxide is used, such as, e.g., 0.1 N sodium hydroxide solution, 0.2 N sodium hydroxide solution, 0.5 N sodium hydroxide solution, 1.0 N sodium hydroxide solution, 1.5 N sodium hydroxide solution, 2.0 N sodium hydroxide solution, or any other suitable solution for providing an amount sufficient of the sodium hydroxide to the composition. In one embodiment, the composition comprises about 1% to about10% 0.1 N sodium hydroxide.

In another embodiment, the pharmaceutical composition includes about 0.5% to about 10% testosterone; about 30% to about 98% alcohol, for example, ethanol or isopropanol; about 0.1% to about 5% isopropyl myristate; about 0.1% to about 5% of a gelling agent; and the balance water. The percentages of components are weight to weight of the composition. In one embodiment, the composition comprises about 1% to 10% 0.1 N sodium hydroxide.

In yet another embodiment, the pharmaceutical composition includes testosterone in a hydroalcoholic gel. The testosterone may be present in a concentration of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about10%, about 10.1%, about 10.2%, about 10.3%, about 10.4%, about 10.5%, about 10.6%, about 10.7%, about 10.8%, about 10.9%, about 11%, about 11.1%, about 11.2%, about 11.3%, about 11.4%, about 11.5%, about 11.6%, about 11.7%, about 11.8%, about 11.9%, about 12%, about 12.1%, about 12.2%, about 12.3%, about 12.4%, about 12.5%, about 12.6%, about 12.7%, about 12.8%, about 12.9%, about 13%, about 13.1%, about 13.2%, about 13.3%, about 13.4%, about 13.5%, about 13.6%, about 13.7%, about 13.8%, about 13.9%, about 14%, about 14.1%, about 14.2%, about 14.3%, about 14.4%, about 14.5%, about 14.6%, about 14.7%, about 14.8%, about 14.9%, or about 15% weight to weight of the composition. The enhancer in this embodiment includes isopropyl myristate, which may be present in a concentration of about 0.5%, about 0.65%, about 0.75%, about 0.85%, about 0.95%, about 1%, about 2%, about 3%, about 4%, or about 5% weight to weight of the composition. The pharmaceutical composition also includes a C1-C4 alcohol present in a concentration of about 70%l, about 71%, about 71.4%, about 71.8%, about 72%, about 72.3%, about 72.5%, about 72.7%, about 73%, about 73.5%, about 74%, about 74.5%, about 75% or about 75% weight to weight of the composition. Further, the pharmaceutical composition includes polyacrylic acid and/or carboxymethylcellulose as the gelling agent. In one embodiment, the gelling agent is polyacrylic acid present in a concentration of about 1% weight to weight of the composition.

One such testosterone gel has only recently been made available in the United States under the trademark AndroGel® by Unimed Pharmaceuticals, Inc., Marietta, Ga., the assignee of this application. In one embodiment, the gel is comprised of the following substances in approximate amounts:

TABLE 3
Composition of AndroGel ®

| | AMOUNT (w/w) | |
|---|---|---|
| SUBSTANCE | PER 100 g OF GEL | |
| Testosterone | 1.0 | g |
| Carbopol 980 | 0.90 | g |
| Isopropyl myristate | 0.50 | g |
| 0.1 N NaOH | 4.72 | g |
| Ethanol (96% v/v) | 71.4 | g* |
| Purified water (qs) | to 100 | g |

*Corresponding to 67 g of ethanol

One skilled in the art will appreciate that the constituents of this formulation may be varied in amounts yet continue to be within the spirit and scope of the present invention. For example, the composition may contain about 0.1 to about 10.0 g of testosterone, about 0.1 to about 5.0 g CARBOPOL, about 0.1 to about 5.0 g isopropyl myristate, and about 30.0 to about 98.0 g ethanol.

In still another embodiment, the composition comprises testosterone in an amount greater than 0.01%, a penetration enhancing agent in an amount greater than about 0.1%, a thickening agent in an amount greater than about 0.1%, and a lower alcohol in an amount greater than about 30% w/w of the composition.

The gel is rubbed or placed onto an area of skin of the subject and allowed to dry. Illustratively, the gel is rubbed onto an area of skin, for example, on the upper outer thigh and/or hip once daily. Following application the subject washes his or her hands. Application of the gel results in an increased testosterone level having a desirable pharmacokinetic profile and is effective to treat or prevent bone deterioration or osteoporosis, or the symptoms associated with, or related to bone deterioration or osteoporosis in the subject. The composition is thus useful for treating a number of bone disorders, conditions or diseases in both men and women.

In one embodiment, the present invention employs a packet having a polyethylene liner compatible with the components of a testosterone gel, as described below. The packet may hold a unit dose or multiple dose.

In another embodiment, the methods and compositions employ a composition that is dispensed from a rigid multi-dose container (for example, with a hand pump) having a larger foil packet, for example, of the composition inside the container. Such larger packets can also comprise a polyethylene liner as above. In one embodiment, the multi-dose container comprises an airless pump that comprises a polyethylene pouch within a canister with a hand pump inserted. In one embodiment, the polyethylene pouch comprises 44 g or 88 g of product. In one embodiment, the pump is primed before use, such as, e.g., by fully depressing the pump three times and discarding the gel. In one embodiment, the pump contains enough product to allow for priming and a set number of precise doses. In one embodiment, each full pump depression delivers 1.25 g of testosterone gel. In this embodiment, a 3.75 g dose of gel would require 3 pump depressions. A 5 g dose of gel would require 4 pump depressions. A 7.5 g dose of gel would require 6 pump depressions. A 10 g dose of gel would require 8 depressions, and so on. Of course, each pump depression can deliver any amount of testosterone gel suitable for delivering the desired dose.

It has been shown, and is discussed in U.S. Pat. No. 6,503,894, U.S. Published Patent Applications 2002/0183296, 2003/0022877, 2003/0050292, 2003/0139384, 2003/0232072, 2004/0002482, 2004/0092494, and U.S. patent application Ser. Nos. 09/703,753, 10/787,071, 10/825,540, 10/828,678, 10/829,618, 10/867,435, 10/924,421, and 10/925,421, herein incorporated by reference in their entirety, that transdermal application of testosterone using AndroGel® to hypogonadal men results in improved testosterone levels, mood, libido and sexual performance. As disclosed herein, it has now been discovered that AndroGel® may also be used for the treatment or prevention of bone disorders, conditions, or diseases, such as, e.g., bone deterioration or osteoporosis.

The methods and compositions of the present invention provide enhanced treatment options for treating, preventing, reversing, halting or slowing the progression of bone deterioration, such as, e.g., deterioration of the trabecular architecture, or osteoporosis in a subject, for example, a man, as compared to those currently available.

In one embodiment, the pharmaceutical composition of the present invention is administered once, twice, or three times a day, or as many times necessary to achieve the desired therapeutic effect. In another embodiment the composition of the present invention is administered once, twice, or three times a day on alternate days. In another embodiment the composition of the present invention is administered once, twice, or three times a day on a weekly, biweekly, or monthly basis.

Besides being useful for human treatment, the present invention is also useful for veterinary treatment of mammals, reptiles, birds, exotic animals and farm animals, including mammals, rodents, and the like. In one embodiment, the mammal includes a primate, for example, a human, a monkey, or a lemur, a horse, a dog, a pig, or a cat. In another embodiment, the rodent includes a rat, a mouse, a squirrel or a guinea pig.

In one embodiment of the present invention a method is provided for treating, preventing, or reducing the risk of developing a bone disorder, condition or disease, such as, e.g., bone deterioration or osteoporosis in a subject in need thereof, that is, a subject indicated for having, or at risk of developing the bone disorder, condition or diseases, such as, e.g., bone deterioration or osteoporosis. The method comprises administering a pharmacologically effective amount of a composition to an area of skin of the subject for delivery of testosterone to blood serum of the subject. The composition comprises: about 0.01% to about 15% (w/w) testosterone; about 0.01% to about 50% (w/w) penetration enhancing agent; about 0.01% to about 50% (w/w) gelling agent; about 30% to about 98% (w/w) lower alcohol; and the balance water.

The composition is capable of releasing the steroid after applying the composition to the skin at a rate and duration that delivers in one embodiment of the present invention at least about 10 μg per day of the steroid to the blood serum of the subject.

In another embodiment of the present invention, the composition is capable of releasing the testosterone after applying the composition to the skin of a subject at a rate and duration that achieves a circulating serum concentration of testosterone greater than about 400 nag per dl serum during a time period beginning about 2 hours after administration and ending about 24 hours after administration.

In another embodiment of the present invention, the composition is capable of releasing the testosterone after applying the composition to the skin of a subject at a rate and duration that achieves a circulating serum concentration of the testosterone between about 400 nag testosterone per dl serum to about 1050 nag testosterone per dl serum.

In another embodiment of the present invention, for each about 0.1 gram per day application of the composition of the present invention to the skin of a subject, an increase of at least about 5 ng/dl in serum testosterone concentration results in the subject.

In another embodiment of the present invention, the composition of the present invention is provided to a subject for daily administration in about a 0.1 g to about a 10 g dose. The composition of the present invention can be provided in any suitable dose, such as, e.g., from about 0.1 g to about 10 g, for example, about 0.1 g, about 0.44 g, about 0.88 g, about 1 g, about 1.32 g, about 1.5 g, about 1.75 g, about 2 g, about 2.25 g, about 2.5 g, about 2.75 g, about 3 g, about 3.5 g, about 3.75 g, about 4 g, about 4.25 g, about 4.5 g, about 4.75 g, about 5 g, about 5.25 g, about 5.5 g, about 5.75 g, about 6 g, about 6.25 g, about 6.5 g, about 6.75 g, about 7 g, about 7.25 g, about 7.5 g, about 7.75 g, about 8 g, about 8.25 g, about 8.5 g, about 8.75 g, about 9 g, about 9.25 g, about 9.5 g, about 9.75 g, about 10 g, or any other suitable dose.

In one embodiment of the invention, a 3.75 g dose of the composition of the present invention contains 37.5 mg of testosterone, a 5 g dose of the composition of the present invention contains 50 mg of testosterone, a 7.5 g dose of the composition of the present invention contains 75 mg, and a 10 g dose of the composition of the present invention contains 100 mg of testosterone.

In yet another embodiment of the present invention, the subject in need of treatment has a serum total testosterone level before the first application (pretreatment) of the composition of the present invention of less than about 300 ng/dl.

In another embodiment of the present invention, where after at least about 30 days of daily administration of the composition of the present invention the serum testosterone concentration in a subject, is at least about 300 ng/dl to about 1050 ng/dl, such as, for example, about 400 ng/dl to about 1050 ng/dl, about 500 ng/dl to about 1050 ng/dl, about 600 ng/dl to about 1050 ng/dl, or about 700 ng/dl to about 1050 ng/dl.

In still another embodiment of the present invention, where after daily administration of the composition of the present invention the total testosterone concentration in a subject is greater than about 300 ng/dl. In one embodiment, the total serum androgen concentration in the subject is greater than about 400 ng/dl, about 500 ng/dl, about 600 ng/dl or about 700 ng/dl. In one embodiment, the total testosterone concentration is measured after 24 hours of administration. In one embodiment, the total testosterone concentration is measured after 2 days of daily administration, such as, for example, after 10 days, 20 days, or 30 days.

In another embodiment of the methods, kits, combinations, and compositions of the present invention, the composition of the present invention is administered once, twice, or three times daily to a subject for at least about 7 days. In one embodiment, the composition is administered once a day.

The present invention also provides a method of treating, preventing or reducing the risk of developing bone deterioration or osteoporosis in a subject in need thereof, that is, a subject indicated for having, or at risk of developing bone deterioration or osteoporosis, by administering to the subject an amount of a composition comprising: about 0.5% to about 10% (w/w) testosterone; about 0.1% to about 5% (w/w) penetration enhancing agent; about 0.1% to about 5% (w/w) thickening agent; about 30% to about 98% (w/w) lower alcohol; and the balance water.

The present invention also provides a method for treating, preventing, or reducing the risk of developing bone deterioration or osteoporosis in a subject comprising: administering to a subject in need thereof an effective amount of a pharmaceutical composition comprising: about 0.1% to about10% (w/w) testosterone; about 0.1% to about 5% (w/w) isopropyl myristate; about 0.1% to about 5% (w/w) thickening agent; about 30% to about 98% (w/w) lower alcohol; and the balance water. In one embodiment, the thickening agent is polyacrylic acid, such as, Carbopol® and the composition further comprises a hydroxide releasing agent, such as, e.g., sodium hydroxide. In one embodiment, the percentages do not add up to 100% and the composition further comprises water q.s. to 100%.

Achieving target delivery rates demonstrated by testosterone gel can be estimated from the pharmacokinetics in testosterone gel in men. The mean serum concentration (Cavg) values in men after applying of varying amounts of gel to the upper body is given in the Table below.

TABLE 4
Mean Average Serum Testosterone Concentrations and Daily Delivery
Rate after Administration of Testosterone Gel 1% in Men

| Dose (μL) | Mean Cavg | Daily Delivery Rate |
|---|---|---|
| (gram) | (ng/dL) | (μg/day)^{a} |
| 5.0 | 555 (±225) | 3330 |
| 7.5 | 601 (±309) | 3606 |
| 10 | 713 (±209) | 4278 |

^{a}Metabolic Clearance Rate of Daily Testosterone = 600 L/day

Based on the results obtained in men, a testosterone gel dose of 0.5 grams delivers approximately 300 μg of testosterone per day.

The present invention is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

### EXAMPLES

### Example 1

### Administration of 1% Testosterone Gel Treats Bone Deterioration in Hypogonadal Men

This example demonstrates that administration of 1% hydroalcoholic testosterone gel decreases bone deterioration in hypogonadal men.

### Study Design

Ten men with severe and untreated hypogonadism due to known disease were entered into the study. The subjects had severely subnormal early morning serum testosterone concentrations on at least two occasions (mean, 88 ng/dL [3.1 nmol/L]) as a consequence of documented pituitary or hypothalamic disease and had received no testosterone treatment for at least four years before enrollment. All ten subjects had secondary hypogonadism. Eight subjects had pituitary adenomas, one had pinealoma, and one had Kallmann's syndrome. Hypogonadism developed in adulthood in nine. The tenth subject, a 46 year old patient with Kallmann's, was treated with testosterone enanthate from age 15 to 25 before discontinuing it. The estimated duration of hypogonadism was 2-30 years (median, 5 years). Eight of the subjects had never been treated with testosterone. Of the two who had been treated, one had not been treated for four years and the other for 20 years before entering the study. The median age at entry was 51 years (range 31-78).

Ten matched eugonadal men were also recruited. They were required to have a serum testosterone concentration >300 ng/dL (10.4 nmol/L) early in the morning on two occasions and a normal bone mineral density (BMD) of the spine for age (Z-score +2 to −2). Each eugonadal subject was matched exactly for race and within ten years for age to a hypogonadal subject. The median age for the eugonadal subjects at entry was 54 years (range, 28 to 74).

Men with a dietary calcium intake greater than 750 mg/day, as determined by a food frequency questionnaire, or who had any disease or took any medication that could affect bone or who consumed more than two alcoholic beverages a day were excluded.

### Treatment

The testosterone preparation provided as treatment to the hypogonadal men was AndroGel® (Unimed Pharmaceuticals, Inc. Marietta Ga.), a hydroalcoholic gel containing 1% testosterone. The initial dose was 5 g of Androgel (50 mg of testosterone), which the subjects self-administered once a day. The serum testosterone concentration was measured at 1, 3, 6, 12, 18 and 24 months. The dose of Androgel® was increased as high as 10 g a day to maintain a serum testosterone concentration within the normal range for eugonadal men (400-900 ng/dL) throughout the 24 months of the study.

Eugonadal subjects did not receive testosterone or other treatment but had a second determination of the serum testosterone concentration at 24 months.

### Study Protocol

All subjects were assessed by dual energy X-ray absorptiometry (DXA) and magnetic resonance microimaging (μMRI) at 0, 6, 12 and 24 months. The hypogonadal subjects were also assessed for markers of bone metabolism at 0, 3, and 6 months.

### Bone Mineral Density

Bone mineral density (BMD) of the anterior-posterior lumbar spine (L1-L4) and of the right hip was determined by DXA using Hologic densitometers (Hologic, Inc., Bedford, Mass.), a QDR-4500A for the first year of the study and a Dephi A for the second. The Delphi A gave spine values 1% lower, so those values were multiplied by 1.01. Scans from the same subject were evaluated by the “compare” feature of the DXA instrument. The coefficients of variation for long-term instrument stability, as assessed by daily measurements of a phantom, were <0.9%.

### Markers of Bone Metabolism

After an overnight fast, blood and a timed 2-hour urine sample were collected. Serum and urine samples were frozen at −70° C. The markers and methods of assay were as follows: bone-specific alkaline phosphatase (BAP), immunoradiometric assay (Tandem-R Ostase, Beckman-Coulter, Inc., Fullerton, Calif.); osteoprotegerin, enzyme-linked immunosorbent assay (American Laboratory Products Company, Windham, N.H.); intact N-terminal propeptide of type I procollagen (PINP), radioimmunoassay, (Orion Diagnostica UniQ, IDS, Inc. Fountain Hills, Ariz.); cross-linked N-telopeptides of type I collagen (NTx), enzyme-linked immunosorbent assay (Osteomark, Ostex International, Inc. Seattle, Wash.). For each assay, all samples were measured in a single assay run. Intraassay coefficients of variation for these assays were all <10%.

### Testosterone and Estradiol

Serum testosterone was measured by a chemiluminescent enzyme immunoassay (Immulite 2000, Diagnostic Products Corporation, Los Angeles, Calif.). Estradiol was measured by ultrasensitive immunoradiometric assay (DSL, Webster, Tex.). Intra- and interassay coefficients of variation were <10%.

### Magnetic Resonance Microimaging (μUMRI)

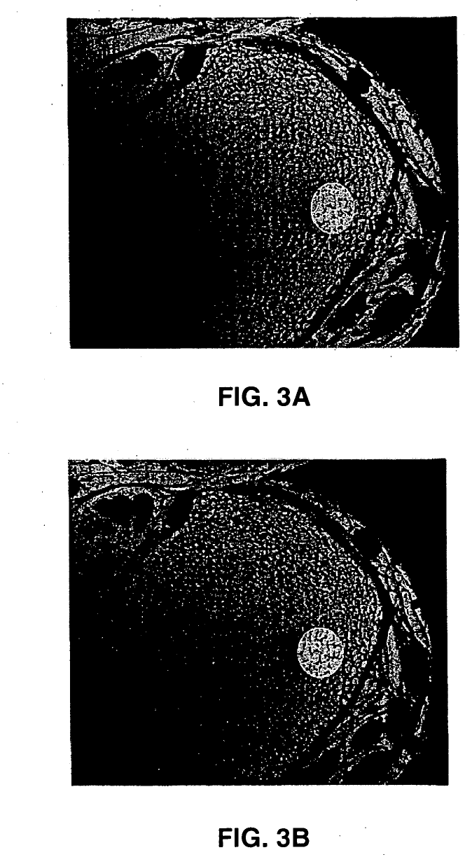

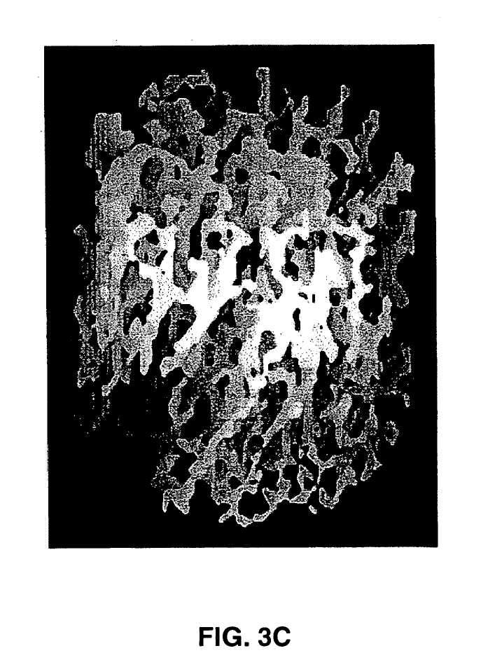

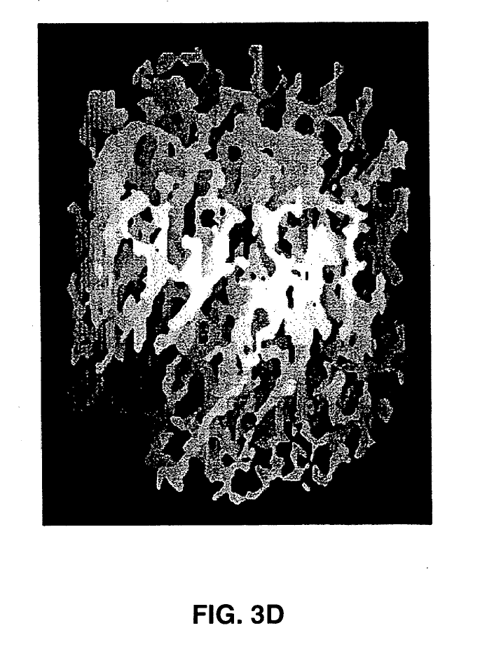

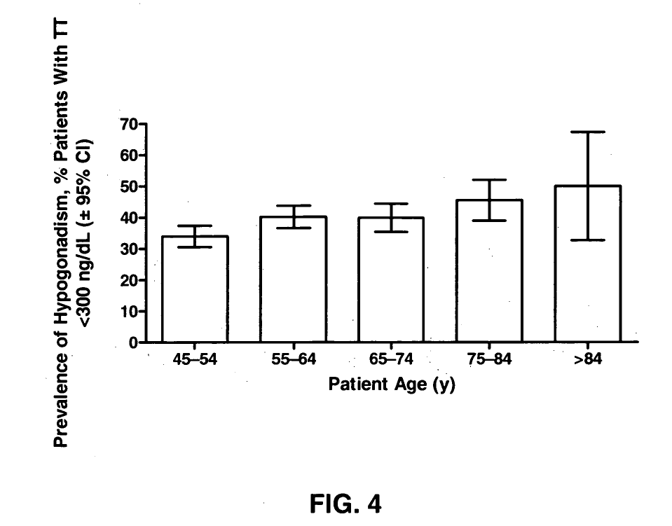

Magnetic resonance microimaging (μMRI) of the right distal tibia was performed using a Signa 1.5 Tesla MR scanner (GE Medical Systems, Milwaukee, Wis.) and a custom-designed receive-only radiofrequency phased array surface coil. The coil was placed on the anterior right tibia, the edge 1 cm proximal to the midpoint of the medial malleolus, and the right foot was immobilized. Twenty-eight images were obtained to map trabecular architecture. Song H K, et al., Magn. Reson. Med., 41:947-53 (1999). The acquisition voxel size was 137×137×410 μm³. The data were processed using a custom-designed program written in IDL (Interactive Data Language, Boulder, Colo.). Wehrli F W, et al., J. Bone Miner. Res., 16:1520-31 (2001). The data were motion-corrected, filtered and Fourier transformed. To ensure that the same volume was analyzed at each time point for each subject, the images corresponding to the 0, 6, 12, and 24 month examinations of each subject were matched for volume of interest by identifying similar trabecular features. After the volume of interest was selected manually on each of the matched slices by tracing a line approximately 1 mm from the endocortical boundary on the anterior half of the distal metaphysis, bone volume fraction maps were computed. Subvoxel processing yielded a voxel size of 69×69×103 μm³. Hwang S N, et al., Magn. Reson. Med., 47 (2002). A cylindrical core measuring 6.85 mm in diameter was obtained from the center of the volume of interest. (FIG. 3).

Digital topological analysis of the trabecular network was performed on the entire volume of interest. Gomberg BR, et al., IEEE Trans. Med. Imaging, 19:166-74 (2000). The topological class of each image voxel was determined, yielding the density of surface voxels, curve voxels, and voxels part of mutual junctions. The topological analysis began with binarization of the three-dimensional images, followed by skeletonization, which converted the plate-like elements of the trabecular network to surfaces and the rodlike elements to curves. Each voxel was then classified as belonging to a surface, curve, or junction between these voxel types. In addition to the simple topological parameters, two composite parameters that have been found to be sensitive to bone loss were calculated. One composite parameter was the surface-to-curve ration, the ratio of all surface voxels to all curve voxels. The higher the ratio, the more intact in the trabecular network, and vice versa. The second composite parameter was the topological erosion index, a ratio of parameters that are expected to increase upon trabecular deterioration (Curve edge and curve interior voxels, surface and profile edges, and curve-curve junction voxels) to those expected to decrease (surface interior voxels and surface-surface junctions). The lower the topological erosion index, the smaller the degree to which the trabecular network has deteriorated. Trabecular thickness was determined by an independent program based on the concept of fuzzy distance transform. Saha P K, et al., IEEE Trans. Med. Imaging, 23:53-62 (2004).

Reproducibility of the MR parameters was determined by calculating the coefficients of variation in eight eugonadal subjects evaluated at 0 and 6 months. The mean values were 2.3% for BVF, 0.4% for trabecular thickness, 6.7% for surface/curve ration, and 4.3% for erosion index.

### Statistical methods

For all parameters, changes from 0 to 24 months were tested by multivariate analysis of variance, one factor repeated measures design. If the change from 0 to 24 months was significant, pair-wise comparisons of each treatment time (6, 12, and 24 months) against pretreatment were determined with Dunnett's test. Missing values were imputed by the method of last observation carried forward. A type I error rate of 0.05 was used for determining statistical significance. SAS statistical software, version 9.1 (SAS Institute, Inc., Cary, N.C.) was used for all analysis.

### Results

All ten hypogonadal subjects completed the 24 months of study. Two missed the six-month visit. One eugonadal subject did not complete the 24 month visit.

### Serum Testosterone and Estradiol Concentrations, Calcium Intake, and Body Mass Index (BMI)

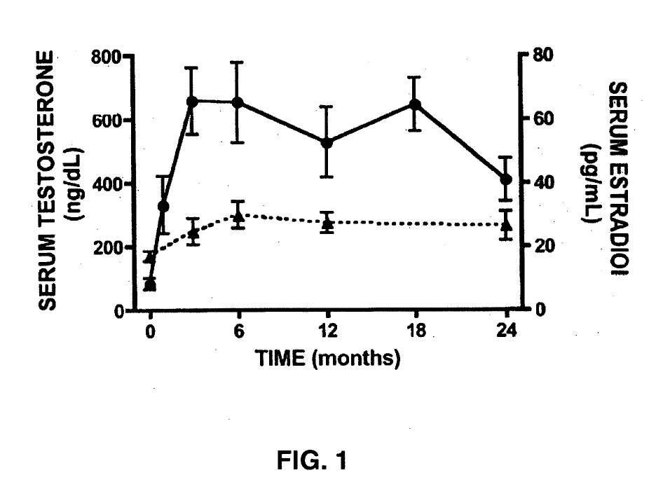

The serum testosterone concentration was markedly subnormal in hypogonadal men before treatment (88±51 ng/dL [3.1±1.8 nmol/L]), rose strikingly to become mid-normal by month 3 of testosterone treatment (656±332 ng/dL [22.8±11.5 nmol/L]), and remained normal throughout the 24 months of treatment (FIG. 1). The serum estradiol concentration was 17±5 pg/mL (62.4±18.4 pmol/L) at baseline, increased to 25±13 pg/mL (91.8±47.7 pmol/L) by month 3 of testosterone treatment, and remained at that level (FIG. 1).

Calcium intake remained normal in hypogonadal subjects during the 24 months of testosterone treatment, 1175±248 mg/day (mean±SD) at 0 months and 1066±375 mg/day at 24 months (P=0.7). BMI also did not change, and was 30.3±3.2 (mean±SD) at 0 months and 30.8±2.1 at 24 months (P=0.8).

The mean serum testosterone concentration was normal in the eugonadal subjects at the beginning of the study (522±126 ng/dL [18.1±4.4 nmol/L]) and remained normal at the end of the study (423±±101 ng/dL [14.7±3.5 nmol/L]).

### Bone Mineral Density

In the ten hypogonadal subjects, bone mineral density (BMD) increased significantly at the anterior-posterior spine (7.4%; P<0.001), total hip, trochanter, and intertrochanteric region during 24 months of testosterone treatment (Table 5).

TABLE 5
Bone Mineral Density (BMD) by DXA (g/cm²) Before and After
24 Months of Testosterone Treatment

| | Time | | Change | % Change | |
|---|---|---|---|---|---|
| Parameter | 0 Months | 24 Months | 0-24 months | 0-24 months | P value¹ |
| Spine BMD | 0.932 ± 0.200 | 1.000 ± 0.219 | 0.069 ± 0.053 | 7.4 ± 5.1 | <0.001 |
| Total Hip BMD | 0.960 ± 0.166 | 0.994 ± 0.162 | 0.034 ± 0.029 | 3.8 ± 3.4 | 0.008 |
| Trochanter BMD | 0.713 ± 0.138 | 0.740 ± 0.136 | 0.027 ± 0.030 | 4.0 ± 4.7 | 0.04 |
| Intertrochanteric | 1.157 ± 0.204 | 1.206 ± 0.205 | 0.049 ± 0.037 | 4.4 ± 3.7 | 0.004 |
| BMD | | | | | |
| Femoral Neck | 0.812 ± 0.140 | 0.828 ± 0.145 | 0.016 ± 0.038 | 2.1 ± 5.1 | 0.3 |
| BMD | | | | | |
| Ward's Triangle | 0.588 ± 0.170 | 0.616 ± 0.172 | 0.028 ± 0.060 | 5.1 ± 10.1 | 0.06 |
| BMD | | | | | |

¹P values were determined by multivariate analysis of variance, one factor repeated measures design using data from all four observation times, 0, 6, 12, and 24 months.

In eugonadal men, BMD did not change significantly at any site during 24 months of observation (Table 6).

TABLE 6
Bone Mineral Density (BMD) by DXA (g/cm²) in Ten
Eugonadal Subjects Followed for 24 Months

| | Time | | Change | % Change | |
|---|---|---|---|---|---|
| Parameter | 0 Months | 24 Months | 0-24 months | 0-24 months | P value¹ |
| Spine BMD | 1.125 ± 0.218 | 1.174 ± 0.245 | 0.022 ± 0.057 | 1.7 ± 4.5 | 0.3 |
| Total Hip BMD | 1.047 ± 0.131 | 1.044 ± 0.123 | −0.003 ± 0.035 | −0.1 ± 3.2 | 0.008 |

¹P values were determined by multivariate analysis of variance, one factor repeated measures design using data from all four observation times, 0, 6, 12, and 24 months.

### Markers of Bone Metabolism

Mean serum intact N-terminal propeptide of type I procollagen (PINP) increased from baseline (41.4±18.1 μg/L) to 3 months (71.3±32.1 μg/L; p=0.02) and then decreased from 3 to 6 months (36.0±21.1 μg/L) of treatment (p=0.01) but bone-specific alkaline phosphatase (BAP) did not change. Serum osteoprotegerin decreased slightly from baseline (4.72±1.28 pmol/L) to 6 months (4.29±0.82 pmol/L), which was of borderline significance (p=0.05). Urine NTx did not change from baseline to 6 months of treatment.

### Magnetic Resonance Microimaging (μMRI)

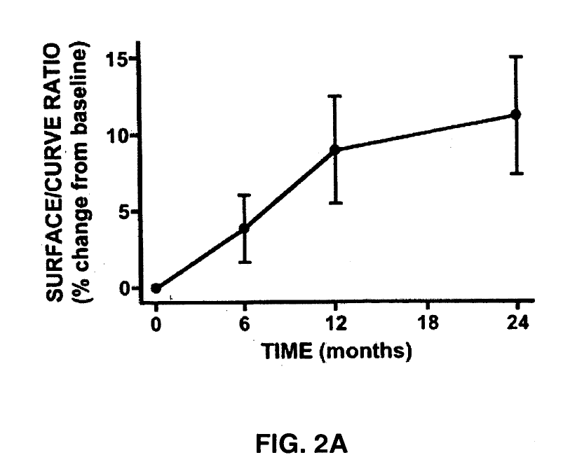

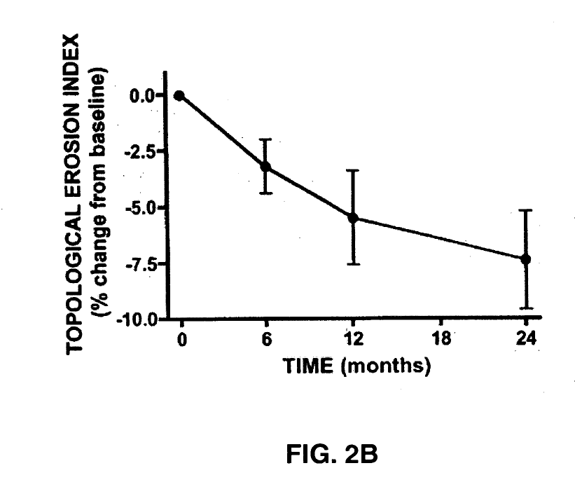

Both magnetic resonance microimaging (μMRI) parameters of the integrity of the trabecular network improved significantly when the hypogonadal subjects were treated with testosterone for 24 months. The surface-to-curve ratio, a ratio of all surface voxels (representing plates) to all curve voxels (representing rods), increased by 9% (P=0.02; multivariate analysis of variance, one way repeated measures, followed by Dunnett's test) after 12 months of testosterone treatment and by 11.2% (P=0.004) after 24 months (FIG. 2, Table 7).

TABLE 7
Architectural Parameters by Magnetic Resonance Microimaging
Before and After 24 Months of Testosterone Treatment

| | Time | | Change | % Change | |
|---|---|---|---|---|---|
| Parameter | 0 Months | 24 Months | 0-24 months | 0-24 months | P value¹ |
| Bone Volume | 0.099 ± 0.013 | 0.103 ± 0.011 | 0.005 ± 0.004 | 5.0 ± 4.2 | <0.001 |
| Fraction | | | | | |
| Trabecular | 118.8 ± 3.6 | 120.5 ± 3.8 | 1.7 ± 0.6 | 1.5 ± 0.5 | <0.001 |
| Thickness (μ) | | | | | |
| Surface-to-Curve | 6.3 ± 1.6 | 7.0 ± 1.5 | 0.6 ± 0.7 | 11.2 ± 11.5 | 0.004 |
| Ratio | | | | | |
| Topological | 1.32 ± 0.28 | 1.22 ± 0.25 | −0.11 ± 0.10 | −7.5 ± 6.7 | 0.002 |
| Erosion Index | | | | | |

¹P values were determined by multivariate analysis of variance, one factor repeated measures design using data from all four observation times, 0, 6, 12, and 24 months.

The topological erosion index, a ratio of topological parameters expected to increase upon trabecular deterioration to those expected to decrease, decreased by 5.6% (P=0.02) after 12 months and by 7.5% (P=0.004) after 24 months of testosterone treatment. (FIG. 2, Table 7). The mean bone volume fraction (BVF), the fractional occupancy of voxels by bone, increased significantly, 5% from 0 to 24 months, and the trabecular thickness increased significantly, starting at 6 months of treatment, almost 2%, during treatment. (Table 7).

FIGS. 3a, 3b, 3c and 3d illustrate that this μMRI technique can identify the same area of the tibia at 24 months as prior to treatment. The top panels show the same cross-sectional areas of the tibia at 0 and 24 months in a single subject. The bottom panels (FIGS. 3c and 3d) show surface projection images, taken from the areas identified by the circles in the top panels (FIGS. 3a and 3b) in this subject and illustrate similar architectural features in both. FIG. 3d also shows a more plate-like architecture after 24 months of testosterone treatment than prior to treatment in this subject (FIGS. 3c), who had the greatest qualitative improvement in topological parameters of the 10 subjects (surface-to-curve ratio increased 33% and topological erosion index decreased 22%).

In the eugonadal subjects, none of the μMRI parameters changed significantly from 0 to 24 months (Table 8).

TABLE 8
Architectural Parameters by Magnetic Resonance Microimaging
in Ten Eugonadal Subjects Followed for 24 Months

| | Time | | Change | % Change | |
|---|---|---|---|---|---|
| Parameter | 0 Months | 24 Months | 0-24 months | 0-24 months | P value¹ |
| Bone Volume | 0.118 ± 0.007 | 0.118 ± 0.009 | −0.001 ± 0.003 | −0.5 ± 2.8 | 0.6 |
| Fraction | | | | | |
| Trabecular | 123.1 ± 2.8 | 123.0 ± 2.5 | −0.1 ± 0.7 | −0.1 ± 0.6 | 0.8 |
| Thickness (μ) | | | | | |
| Surface-to-Curve | 11.0 ± 2.3 | 10.5 ± 2.1 | −0.5 ± 1.4 | −4.1 ± 12.3 | 0.5 |
| Ratio | | | | | |
| Topological | 0.89 ± 0.16 | 0.92 ± 0.14 | 0.03 ± 0.08 | 4.1 ± 9.1 | 0.5 |
| Erosion Index | | | | | |

¹P values were determined by multivariate analysis of variance, one factor repeated measures design using data from all four observation times, 0, 6, 12, and 24 months.

This example demonstrates that testosterone treatment of hypogonadal men treats bone deterioration, including deterioration of trabecular architecture. Surprisingly, the magnetic resonance microimaging showed a dramatic increase in the μMRI parameters that reflect trabecular architecture, the surface-to-curve ratio and the topological erosion index. These parameters improved dramatically and to a great degree than did other μMRI parameters, including trabecular thickness and bone volume fraction, and to a greater degree than bone mineral density of the spine and hip.

This example further demonstrates that testosterone replacement of hypogonadal men not only retards bone resorption, but may also reverse the deterioration of bone, including deterioration of trabecular architecture. The increase in the surface-to-curve ratio, which is the topologic representation of the ratio of trabecular plates to rods, suggests that testosterone replacement partially restored trabecular connectivity. If testosterone had merely retarded bone resorption and allowed filling of bone resorption cavities, it would have been expected that an increase in μMRI parameters of trabecular thickness and bone volume fraction and an increase in bone mineral density would be observed, but not in surface-to-curve ratio or topological erosion index. In fact, the surface-to-curve ratio and topological erosion index improved to greater degrees than these other parameters. The significance of an improvement in trabecular architecture is that architecture contributes to bone's strength and resistance to fracture, independent of bone volume or density, as demonstrated by several in vitro studies. Hwang S N, et al., Med. Phys., 24:1255-61 (1997); Majumdar S, et al., J. Bone Miner. Res., 12:111-8 (1997); Gordon C L, et al., Canad. Assoc. Radiol. J., 49:390-7 (1998); Oden Z M, et al., Calcif. Tissue Int., 63:67-73 (1998); Ulrich D, et al., Bone, 25:55-60 (1999).

One limitation of the study in this Example is that assessment of trabecular architecture by μMRI had to be performed at a surrogate site, the distal tibia, in order to achieve the resolution sufficient to discern individual trabeculae. This site, however, is rich in trabecular bone, like common sites of osteoporotic fractures, such as the spine and hip, and is also weight-bearing. Another limitation was the lack of a placebo control group, because such severely hypogonadal men could not be allowed to go untreated for 24 months. The marked improvement in architectural parameters seen in the hypogonadal men, however, probably cannot be attributed to a change in the measurement technique, since the ten matched eugonadal men followed simultaneously for 24 months showed no changes. The improvement in architectural parameters also cannot be attributed to changes in the subjects' calcium intake and BMI, which were quite similar at the beginning and the end of the study.

Therefore, this example demonstrates that physiologic replacement of testosterone not only treats bone deterioration by increasing the amount of bone, but also improves parameters of trabecular architecture associated with bone strength.

### Example 2

### Prevalence Rate of Hypogonadism Among Men in a Primary Care Practice Setting

This example demonstrates the prevalence rate of hypogonadism in men aged at least 45 years who present to primary care offices, regardless of the reason for the visit. This example also estimates the age-associated prevalence of hypogonadism and its associated signs and symptoms, as well as whether the occurrence of hypogonadism and reported signs and symptoms of hypogonadism varied between younger (45-64 years) and older (≧65 years) men.

### METHODS

Study Design: The study Was a cross-sectional survey to determine the prevalence of hypogonadism in patients aged at least 45 years who were seen before noon in primary care offices during a 2-week period. Clinicians from a random sample of 2650 primary care practices throughout the United States were contacted. 130 practices qualified for participation. Men who were seen in a participating physician's office between 8 AM and noon during a 2-week period, regardless of the reason for their visit, were invited to participate in the study.

Inclusion criteria included: age 45 years or older, ability to provide a blood sample, willingness to participate, and the ability to read, speak, and understand English. Exclusion criteria included the inability or unwillingness to sign the informed consent form.

Assessments: All eligible patients had a single morning blood draw (between 8 AM and noon) to test for concentrations of total testosterone (TT), free testosterone (FT), bioavailable testosterone (BAT), and sex hormone-binding globulin (SHBG). All blood tests were analyzed by Esoterix Labs, Austin, Tex.

Demographic characteristics, medical history, social history, and concomitant medications were collected to capture the following information: symptoms associated with hypogonadism and comorbid conditions.

Statistical Analysis: The primary analyses focused on descriptive statistics and prevalence estimation for hypogonadism, defined as TT<300 ng/dL. Prevalence estimates (with 95% confidence interval [CI]) of hypogonadism were also obtained for men aged <65 years versus men aged ≧65 years. A second exploratory analysis was conducted to assess the impact of demographic variables and identify potential risk factors (such as age) that were associated with hypogonadism. Odds ratios and corresponding 95% CIs were determined for age-associated prevalence and for a 10-year increase in age.

### RESULTS

Table 9 sets forth patient characteristics stratified by age.

TABLE 9
Patient Characteristics Stratified By Age

| | Men Aged <65 y | | Men Aged ≧65 y | |
|---|---|---|---|---|
| | n = 1453 | | n = 712 | |
| | Hypogonadal | Eugonadal | Hypogonadal | Eugonadal |
| Characteristic^{a} | n = 537 | n = 915 | n = 299 | n = 411 |
| Race/ethnicity, | | | | |
| n (%) | | | | |
| White | 447 (83.2) | 734 (80.2) | 253 (84.6) | 343 (83.5) |
| Black | 77 (14.3) | 126 (13.8) | 37 (12.4) | 54 (13.1) |
| Hispanic | 11 (2.0) | 33 (3.6) | 4 (1.3) | 9 (2.2) |
| Asian | 1 (0.2) | 8 (0.9) | 1 (0.3) | 3 (0.7) |
| Other | 1 (0.2) | 14 (1.5) | 4 (1.3) | 2 (0.5) |
| Age, y | 54.9 ± 5.5 | 54.2 ± 5.5 | 73.5 ± 6.0 | 72.4 ± 5.8 |
| (mean ± SD) | | | | |
| BMI, kg/m² | 32.4 ± 6.1 | 29.0 ± 5.1 | 29.9 ± 5.7 | 27.6 ± 4.7 |
| (mean ± SD)* | | | | |
| BMI >25 kg/ | 471 (93.4) | 698 (81.2) | 227 (83.7) | 277 (71.6) |
| m², n (%)* | | | | |
| BP, mmHg | | | | |
| (mean ± SD)^{†} | | | | |
| Systolic BP | 132 ± 15.3 | 128 ± 14.9 | 132.8 ± 16.7 | 133.4 ± 17.0 |
| Diastolic BP | 81 ± 9.8 | 80.5 ± 9.5^{†} | 75.2 ± 10.3 | 77.1 ± 9.80 |

BMI = body mass index;
BP = blood pressure;
SD = standard deviation.
^{a}Patient data not available for 1 man aged <65 years and 2 men aged ≧65 y.
*For patients aged <65 years, BMI information was not reported for 33 hypogonadal patients and 56 eugonadal patients. For patients aged ≧65 years, BMI information was not reported for 28 hypogonadal patients and 24 eugonadal patients.
^{†}In patients aged <65 years, BP information was not reported for 1 eugonadal patient.

As shown in Table 10, of 2162 patients enrolled in the study with evaluable testosterone levels, 836 were hypogonadal, indicating a crude prevalence of 38.7%.

TABLE 10
Prevalence of Hypogonadism By Age

| | Hypogonadal Prevalence Rate | Eugonadal Prevalence Rate |
|---|---|---|
| Age (y) | n (%) | n (%) |
| 45-64 | 537 (36.9) | 915 (63.0) |
| ≧65 | 299 (42.1) | 411 (57.9) |

More hypogonadal patients ≦64 years reported

The prevalence of hypogonadism was greater in men aged ≧65 years (42.1%) versus men aged 45-64 years (36.9%). Men ≧65 years of age were 1.24 times more likely (95% CI, 1.03-1.49) to have hypogonadism than men aged 45 to 65 years. The odds of having hypogonadism was 1.17 more likely (95% CI, 1.08-1.27) for every 10 year increase in age. Furthermore, when stratified by total testosterone, free testosterone and bioavailable testosterone were significantly reduced in hypogonadal versus eugonadal men.

Table 11 presents the prevalence of signs and symptoms of hypogonadism in patients stratified by age.

TABLE 11
Signs and Symptoms of Hypogonadism

| | Men Aged <65 y | | Men Aged ≧65 y | |
|---|---|---|---|---|
| | n = 1453 | | n = 712 | |
| Signs/Symptoms, | Hypogonadal | Eugonadal | Hypogonadal | Eugonadal |
| n (%)^{a} | n = 537 | n = 915 | n = 299 | n = 411 |
| Decrease in ability/ | 245 (45.6)* | 304 (33.4) | 108 (36.1) | 240 (58.4) |
| frequency to | | | | |
| perform sexually | | | | |
| Decrease in sexual | 210 (39.1)* | 264 (29.0) | 140 (46.8) | 203 (49.4) |
| desire/libido | | | | |
| Physical exhaustion/ | 143 (26.6)* | 192 (21.1) | 100 (33.4) | 111 (27.0) |
| lacking vitality | | | | |
| Decrease in | 106 (19.7) | 169 (18.6) | 96 (32.1) | 128 (31.1) |
| muscular strength/ | | | | |
| feeling of weakness | | | | |
| Decrease in general | 108 (20.1) | 177 (19.4) | 87 (29.1)* | 82 (20.0) |
| feeling of well- | | | | |
| being | | | | |
| Depressive mood | 90 (16.8) | 150 (16.4) | 48 (16.1) | 56 (13.6) |
| Not reported | 0 | 4 (0.4) | 0 | 0 |

*P ≦ 0.015 obtained from chi-square test testing hypogonadal patients vs eugonadal patients.
^{a}Signs and symptom data not available for 1 man aged <65 years and 2 men aged ≧65 y.

As shown in Table 11, more hypogonadal patients ≦64 years of age reported decreased ability/frequency to perform sexually (P<0.001), decreased sexual desire/libido (P<0.001), and decreased physical exhaustion/lacking vitality than did eugonadal men of the same age range (P=0.015). More hypogonadal men aged ≧65 years experienced a decline in general sense of well-being than did eugonadal men aged ≧65 years (P=0.005) (Table 11).

Table 12 shows the frequency of hypogonadal symptoms in patients separated by age.

TABLE 12
Frequency of Hypogonadal Symptoms and Age

| Number of Signs/ | Men Aged <65 y | | Men Aged ≧65 y | |
|---|---|---|---|---|
| Symptoms of | n = 1453 | | n = 712 | |
| Hypogonadism, | Hypogonadal | Eugonadal | Hypogonadal | Eugonadal |
| n (%)^{a} | n = 537 | n = 915 | n = 299 | n = 411 |
| 0 | 205 (38.2) | 435 (47.7) | 78 (26.1) | 105 (25.5) |
| 1 | 89 (16.6) | 142 (15.6) | 51 (17.1) | 64 (15.6) |
| 2 | 95 (17.7) | 132 (14.5) | 55 (18.4) | 105 (25.5) |
| 3 | 52 (9.7) | 63 (6.9) | 38 (12.7) | 56 (13.6) |
| 4 | 39 (7.3) | 65 (7.1) | 28 (9.4) | 38 (9.2) |
| 5 | 31 (5.8) | 43 (4.7) | 30 (10.0) | 32 (7.8) |
| 6 | 26 (4.8) | 31 (3.4) | 19 (6.4) | 11 (2.7) |

^{a}Signs/Symptom data not available for 1 man aged <65 years and 2 men aged ≧65 y.

The frequency of common signs and symptoms was similar between hypogonadal and eugonadal men across all age groups. A greater percentage of younger men (<65 y) versus older men reported no signs or symptoms of hypogonadism (Table 12).

### CONCLUSION

This example demonstrates that in patients presenting to primary care offices, 42.1% of men aged ≧65 years had low total testosterone concentrations compared with 38.7% for all men in the study aged at least 45 years. This example also demonstrated that a greater percentage of younger (<65 y) versus older (≧65 y) men reported no signs or symptoms of hypogonadism.

All cited literature and patent references are hereby incorporated herein by reference. Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed is:

1. A method of treating, preventing or reducing the risk of developing bone deterioration or osteoporosis in a subject in need thereof, comprising: administering an amount of a hydroalcoholic gel pharmaceutical composition to an area of skin of the subject, which delivers a therapeutically-effective amount of a steroid in the testosterone synthetic pathway to the blood serum of the subject, wherein the composition comprises:

| | |
|---|---|
| a. | about 0.1% to about 10% (w/w) of the steroid in the testosterone |
| | synthetic pathway; |
| b. | about 0.1% to about 5% (w/w) penetration enhancing agent; |
| c. | about 0.1% to about 5% (w/w) thickening agent; |
| e. | about 45% to about 98% (w/w) lower alcohol; and |
| f. | the balance purified water; |

wherein the composition is capable of releasing the steroid after applying the composition to the skin at a rate and duration that delivers at least about 10 μg per day of the steroid to the blood serum of the subject; and the percentages are on a weight to weight basis of the composition.

2. The method of claim 1, wherein the steroid in the testosterone synthetic pathway comprises about 0.1% to about10% testosterone, or a salt, ester, amide, enantiomer, isomer, tautomer, prodrug, or derivative thereof.

3. The method of claim 1, wherein the steroid in the testosterone synthetic pathway comprises about 1% testosterone, or a salt, ester, amide, enantiomer, isomer, tautomer, prodrug, or derivative thereof.

4. The method of claim 2, wherein the penetration enhancing agent comprises about 0.1% to about 5% of a C8-C22 fatty acid, a C8-C22 fatty alcohol, a lower alkyl ester of a C8-C22 fatty acid, a di(lower)alkyl ester of a C6-C22 diacid, a monoglyceride of a C8-C22 fatty acid, a tetrahydrofurfuryl alcohol polyethylene glycol ether, a polyethylene glycol, a propylene glycol, a 2-(2-ethoxyethoxy) ethanol, a diethylene glycol monomethyl ether, an alkylaryl ether of polyethylene oxide, a polyethylene oxide monomethyl ether, a polyethylene oxide dimethyl ether, a dimethyl sulfoxide, a glycerol, an ethyl acetate, an acetoacetic ester, a N-alkylpyrrolidone, a terpene or combinations thereof.

5. The method of claim 4, wherein the penetration enhancing agent is isopropyl myristate.

6. The method of claim 2, wherein the thickening agent comprises about 0.1% to about 5% polyacrylic acid.

7. The method of claim 2, wherein the lower alcohol comprises about 45% to about 90% ethanol or isopropanol.

8. The method of claim 2, wherein the hydroalcoholic gel pharmaceutical composition comprises:

| | |
|---|---|
| a. | about 1% (w/w) testosterone; |
| b. | about 0.9% (w/w) CARBOPOL ®; |
| c. | about 0.5% (w/w) isopropyl myristate; |
| d. | about 67% (w/w) ethanol; and |
| e. | the balance purified water. |

9. The method of claim 2, wherein the composition is capable of releasing the testosterone after applying the composition to the skin at a rate and duration that achieves circulating serum concentration of the testosterone greater than about 400 ng testosterone per dl serum during a time period beginning about 2 hours after administration and ending about 24 hours after administration.

10. The method of claim 9, wherein the serum testosterone concentration is maintained between about 400 ng testosterone per dl serum to about 1050 ng testosterone per dl serum.

11. The method of claim 2, wherein for each about 0.1 gram per day application of the composition to the skin, an increase of at least about 5 ng/dl in serum testosterone concentration results in the subject.

12. The method of claim 2, wherein the composition is provided to the subject for daily administration in about a 0.1 g to about a 10 g dose.

13. The method of claim 2, wherein the amount of the composition is a 5 g dose delivering about 50 mg of testosterone to the skin.

14. The method of claim 2, wherein the amount of the composition is a 7.5 g dose delivering about 75 mg of testosterone to the skin.

15. The method of claim 2, wherein the amount of the composition is a 10 g dose delivering about 100 mg of testosterone to the skin.

16. The method of claim 2, wherein the composition is provided to the subject in one or more packets.

17. The method of claim 16, wherein the packet comprises a polyethylene liner between the composition and inner surface of the packet.

18. The method of claim 2, wherein the subject has a pretreatment serum testosterone concentration less than about 300 ng/dl.

19. The method of claim 18, wherein after at least about 30 days of daily administration serum testosterone concentration in the subject is at least about 300 ng/dl to about 1050 ng/dl.

20. The method of claim 2, wherein the composition is administered once, twice, or three times daily for at least about 7 days.
